(19) United States
(12) Patent Application Publication
(10) Pub. No.: US 2024/0293384 A1
(43) Pub. Date: Sep. 5, 2024

(54) METHODS AND COMPOSITIONS FOR TREATMENT OF COVID-19

(71) Applicant: THE REGENTS OF THE UNIVERSITY OF CALIFORNIA, OAKLAND, CA (US)

(72) Inventors: Malcolm Casale, IRVINE, CA (US); Leslie M. Thompson, IRVINE, CA (US); Thomas E. Lane, IRVINE, CA (US)

(73) Assignee: THE REGENTS OF THE UNIVERSITY OF CALIFORNIA, OAKLAND, CA (US)

(21) Appl. No.: 18/575,739

(22) PCT Filed: Jun. 29, 2022
(86) PCT No.: PCT/US22/73268

§ 371 Date: Dec. 29, 2023

Related U.S. Application Data
(60) Provisional application No. 63/202,907, filed on Jun. 29, 2021.

(51) Int. Cl. A61K 31/4439; A61K 31/4709; A61P 31/14

(52) U.S. Cl. CPC A61K 31/4439; A61K 31/4709; A61P 31/14

(57) ABSTRACT

A drug scaffold, MIND-4 and MIND4-17, and derivatives thereof, can be used to treat and prevent infection by SARS-CoV-2 that causes COVID-19, other coronavirus infections, and the associated immune and inflammatory responses. Surprisingly, these compounds inhibit the tubulin network of virally-infected cells, and inhibit viral replication. The compounds and methods described herein are useful in the inhibition of viral replication in a virally-infected cell. The cell can be a cell infected with severe acute respiratory syndrome corona virus 2 (SARS-CoV-2), or infected with another SARS coronavirus, or SARS-like coronavirus.

This application claims benefit of U.S. provisional patent application No. 63/202,907, filed Jun. 29, 2021, the entire contents of which are incorporated by reference into this application.

### BACKGROUND

Covid-19 is caused by the virus severe acute respiratory syndrome corona virus 2 (SARS-CoV-2), causing a severe acute respiratory syndrome. COVID-19 is related to previous coronavirus human infections, including SARS-CoV and Middle East respiratory syndrome-related coronavirus (MERS-CoV). COVID-19 and the evolving variants has created an international health crisis and drugs that could prevent infection or reduce symptoms without preference for serotype would be of great impact. For SARS-CoV-2, a vast number of studies have shown that in addition to virus propagation, the host inflammatory response is a critical determinant of disease outcome. Similarly, responses to influenza A and B infections also intersect with the host immune system and inflammatory responses.

Nuclear factor erythroid 2 p45-related factor2 (NRF2) is a cellular protein that has anti-oxidant and anti-inflammatory properties and has also recently been implicated as having an inhibitory effect on influenza virus entry and replication¹. NRF2 can protect against influenza A infection of alveolar epithelial cells through its anti-oxidant properties² and compounds that have NRF2 activating and anti-inflammatory properties including sulforaphane (SFN) and epigallocatechin gallate (EGCG)¹ and other molecules (bardoxolonoe methyl and a natural plant product PB125³. Further, NRF2 activators have been demonstrated to have anti-viral properties, including inhibition of virus entry into cells⁴.

The use of NRF2 activators, such as DMF, have been proposed for deployment against SARS-CoV-25 and other related viruses to restore redox and protein homeostasis, promote resolution of aberrant inflammatory responses, including inhibition of NF-kB, apoptosis and expression of Toll-like Receptors, and facilitate repair. NRF2 activity declines with age, consistent with the increased susceptibility of the elderly to COVID-19³. However, in general these compounds have multiple targets and potential off-target effects or low potency.

There remains a need for effective treatment of viral infections, particularly those related to coronavirus infections, and for the associated inflammatory responses, that does not involve adverse and off-target effects.

### SUMMARY

These needs and more are met by use of a drug scaffold, MIND-4 and MIND4-17, and derivatives thereof, to treat and prevent infection by SARS-CoV-2 that causes COVID-19, other coronavirus infections, and the associated immune and inflammatory responses. Surprisingly, these compounds inhibit the tubulin network of virally-infected cells, and inhibit viral replication. Accordingly, described herein are compounds and methods that are useful in the inhibition of viral replication in a virally-infected cell. In some embodiments, the cell is a cell infected with severe acute respiratory syndrome corona virus 2 (SARS-CoV-2). In some embodiments, the cell is a cell infected with another SARS coronavirus, or SARS-like coronavirus.

The compounds and methods described herein can be used to inhibit the tubulin network of virally-infected cells, and to ameliorate symptoms including inflammatory and immune responses, of viral infection, including, for example, infection with SARS-CoV-2. The compounds, as well as various modes of delivery and administration, are described in U.S. Pat. No. 9,737,525. These compounds are referred to herein as MIND4 compounds. In some embodiments, the compound is a derivative of MIND4 referred to as MIND4-17.

In some embodiments, described herein is a method of inhibiting viral replication in a cell infected with severe acute respiratory syndrome corona virus 2 (SARS-CoV-2), the method comprising contacting the infected cell with a compound selected from the group consisting of the MIND4 compounds depicted in FIGS. 1A-1F, or a pharmaceutically acceptable salt of any one of these compounds. In some embodiments, the compound is

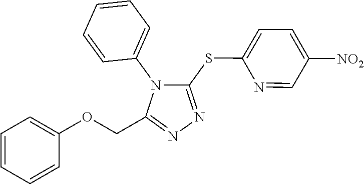 [SMILES: O=[N+]([O-])c1ccc(Sc2nnc(COc3ccccc3)n2-c2ccccc2)nc1]

or a pharmaceutically acceptable salt thereof.

In some embodiments, the cell is a neuron. In some embodiments, the neuron is in the central nervous system of a subject. In some embodiments, the cell is a lung cell. In some embodiments, the cell is ex vivo. In some embodiments, the cell is in vivo.

Also described herein is a method of treating a SARS-CoV-2 infection in a subject, the method comprising administering a therapeutically effective amount of the MIND4 compound recited above (or in FIG. A) to the subject. Additionally described is a method of ameliorating inflammatory and/or immune reactions in a subject infected with a virus, the method comprising administering a therapeutically effective amount of the MIND4 compound to the subject. In some embodiments, the administering is intravenous or intranasal. In some embodiments, the administering is by inhalation.

In some embodiments, the compound is administered at a dose of about 40 to about 80 mg/kg body weight. In some embodiments, the dose is about 60 mg/kg body weight.

The subject is typically a mammal. In one embodiment, the mammal is human. In other embodiments, the mammal is a veterinary subject. Examples of veterinary subjects include, but are not limited to, equine, canine, bovine, porcine, ovine, and feline subjects.

### BRIEF DESCRIPTION OF THE DRAWINGS

FIGS. 1A-1F show MIND4 compounds.

FIGS. 2A-2B show infection of neurons with SARS-CoV-2. To directly test the capability of SARS-CoV-2 to infect neurons, we generated high-purity human iPSC-derived neurons and infected these cells with SARS-CoV-2 (Seattle strain) at a multiplicity of infection (MOI) of 0.1 and the ability of virus to infect cells determined by immunohistochemical staining for anti-nucleocapsid (N) staining of cells. FIG. 2A shows detection of SARS-CoV-2 N protein at both 24 and 48 hours post-infection (hpi) via immunocytochemical staining. FIG. 2B shows that there is an increase in viral antigen by 48 hpi, as determined by counting positive cells/field.

FIG. 3 shows SARS-CoV-2-Spike RNA in HIPSC-neurons treated with DMSO or MIND4, normalized to mock infected neurons 24 and 48 hours post-infection. The data show that treatment of infected human neurons with MIND4-17 (2 uM final concentration) affected viral growth. Neurons were infected with SARS-CoV-2 (MOI=0.1) and treated with either MIND4-17 or vehicle (DMSO) beginning at 24 hpi. RNA was subsequently isolated from infected neurons treated with either DMSO or MIND4-17 at 48 and 72 hpi (24 and 48 hrs post-treatment) and SARS-CoV-2 levels determined by qPCR for Spike mRNA.

FIG. 4 illustrates how the tubulin network is required for SarsCoV2 viral assembly, with the tubulin network shown using IPA network-based analysis. One of the top networks discovered using IPA is this same tubulin network, showing that MIND4-17 causes downregulation of multiple genes in the network (shown in shaded circles) suggesting this as a mechanism of action for the anti-viral properties of MIND4

FIG. 5 shows the effect of treatment with MIND4 versus DMSO control on the human lung epithelial cell line Calu3. Confluent monolayers of cells were incubated at 37° C. in a cell incubation chamber for 24 hrs, at which point SARS-CoV-2 was added to each well and virus allowed to adhere for 1 hr (rocking plates every 15 min) followed by washing and adding media back containing either DMSO) or MIND4-17 (2 μm). Supernatants and cellular RNA were collected at 72 h post-infection to measure viral titers via plaque assay and quantitative PCR (qPCR—using primers specific for Spike RNA), respectively. RNA was also isolated to carry out total RNAseq. MIND4 treatment resulted in a dramatic reduction in viral RNA levels in Calu3-cells when compared to control-treated cells, confirming that MIND4-targeting impacts viral replication.

FIG. 6A is a scatter plot showing that MIND4 infection showed a significant restoration back to uninfected state, as seen on the upper left quadrant where the majority of genes showed reversal from the infected state.

FIG. 6B is a bar graph showing the log 2 ratio numbers of DEGs in treated versus controls.

### DETAILED DESCRIPTION

Demonstrated here for the first time is the discovery that, in two different cell types, MIND4 reduces viral replication, and has an effect on viral mediated cellular gene expression. Disclosed herein are materials and methods that are useful for treatment and prevention of viral infections and virally-infected cells. The surprising discovery of the mechanism of action, that these compounds inhibit the tubulin network of virally-infected cells, indicates the MIND4 compounds can prevent infection by other related viruses, as it is specific for the host factors needed to promote infection, rather than targeting the viral capsid or other specific viral proteins. One of the advantages of the compound is that it targets the host factors required to allow infection by the SARS-CoV-2 and other associated viruses, creating an opportunity for addressing emerging variants of SARS-CoV-2.

### Definitions

All scientific and technical terms used in this application have meanings commonly used in the art unless otherwise specified. As used in this application, the following words or phrases have the meanings specified.

As used herein, a “therapeutically effective” amount of a compound described herein is typically one which is sufficient to achieve the desired effect and may vary according to the nature and severity of the disease condition, and the potency of the compound. It will be appreciated that different concentrations may be employed for prophylaxis than for treatment of an active disease.

As used herein, “compound,” includes all stereoisomers, geometric isomers, and tautomers of the structures depicted. Compounds herein identified by name or structure as one particular tautomeric form are intended to include other tautomeric forms unless otherwise specified.

In some embodiments, a compound provided herein, or salt thereof, is substantially isolated. By “substantially isolated” is meant that the compound is at least partially or substantially separated from the environment in which it was formed or detected. Partial separation can include, for example, a composition enriched in the compound provided herein. Substantial separation can include compositions containing at least about 50%, at least about 60%, at least about 70%, at least about 80%, at least about 90%, at least about 95%, at least about 97%, or at least about 99% by weight of the compound provided herein, or salt thereof. Methods for isolating compounds and their salts are routine in the art.

As used herein, “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein the language “pharmaceutically acceptable carrier” includes saline, solvents, dispersion media, coatings, antibacterial and antifungal agents, isotonic and absorption delaying agents, and the like, compatible with pharmaceutical administration.

The term “pharmaceutically acceptable salt” refers to salts which possess toxicity profiles within a range that affords utility in pharmaceutical applications. Pharmaceutically unacceptable salts may nonetheless possess properties such as high crystallinity, which may render them useful, for example in processes of synthesis, purification or formulation of compounds provided herein. In general the useful properties of the compounds provided herein do not depend critically on whether the compound is or is not in a salt form, so unless clearly indicated otherwise (such as specifying that the compound should be in “free base” or “free acid” form), reference in the specification to a compound provided herein should be understood as encompassing salt forms of the compound, whether or not this is explicitly stated.

As used herein. “contacting” means bringing at least two moieties together, whether in an in vitro system or an in vivo system.

As used herein, “spike protein” or “S-protein” refers to the spike protein of SARS-CoV-2, or a functional portion of spike protein that is sufficient to mediate viral entry.

As used herein, “a” or “an” means at least one, unless clearly indicated otherwise.

As used herein, to “prevent” or “protect against” a condition or disease means to hinder, reduce or delay the onset or progression of the condition or disease.

As used herein, “treating” or “treatment” includes prophylaxis and therapy, and results in amelioration of symptoms, delays progression, or otherwise improves the disease condition of the subject undergoing treatment.

### Compounds

Described herein is a drug scaffold, MIND-4 and MIND4-17, and derivatives thereof, to treat and prevent infection by SARS-CoV-2 that causes COVID-19, other coronavirus infections, and the associated immune and inflammatory responses. These compounds can be used to inhibit the tubulin network of virally-infected cells, and inhibit viral replication. The compounds are thus useful in the inhibition of viral replication in a virally-infected cell.

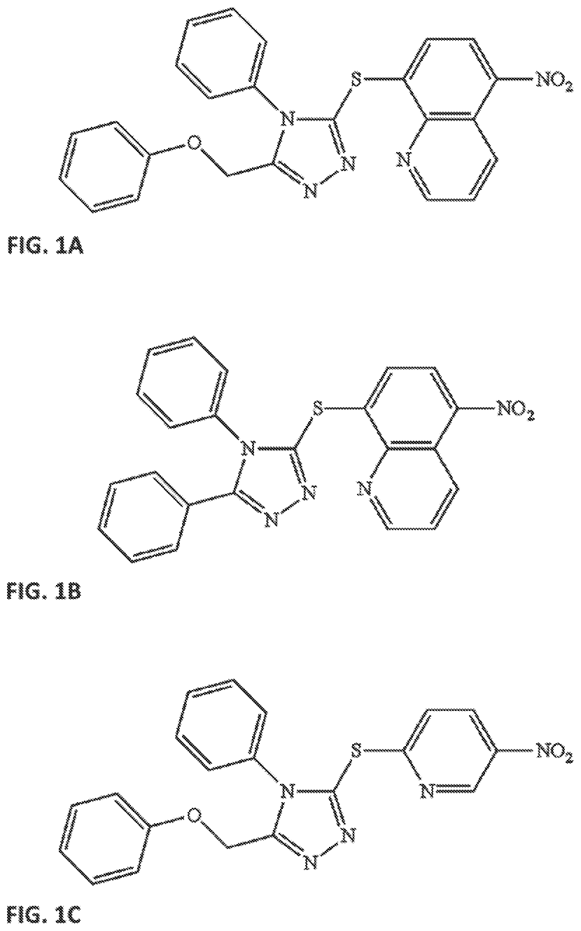

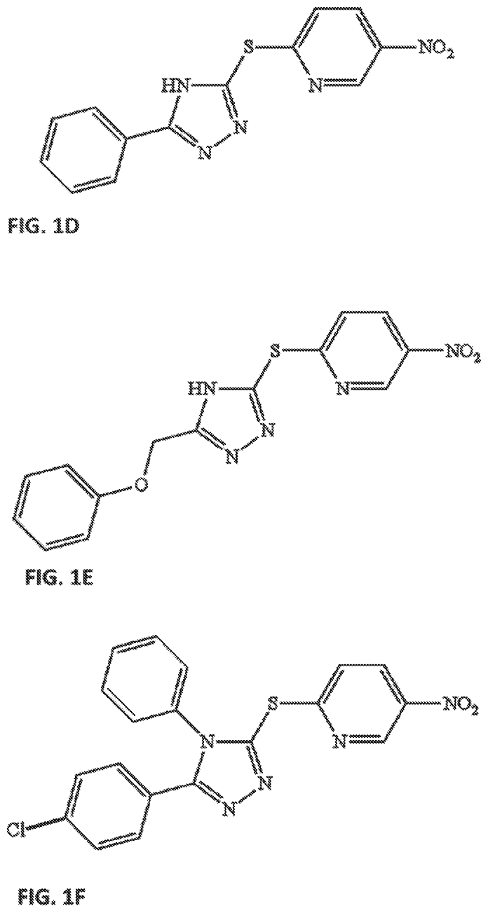

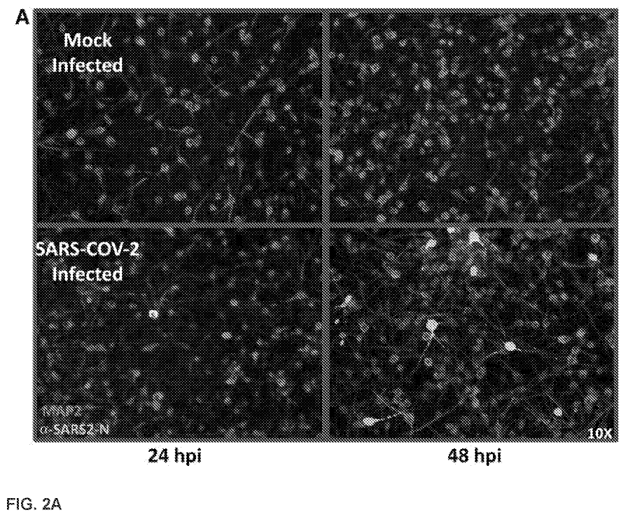

The compounds and methods described herein can be used to inhibit the tubulin network of virally-infected cells, and to ameliorate symptoms including inflammatory and immune responses, of viral infection, including, for example, infection with SARS-CoV-2. The compounds, as well as various modes of delivery and administration, are described in U.S. Pat. No. 9,737,525. These compounds are referred to herein as MIND4 compounds. Representative compounds are provided in FIGS. 1A-1F. In some embodiments, the compound is a derivative of MIND4 referred to as MIND4-17 (5-Nitro-2-{[5-(phenoxymethyl)-4-phenyl-4H-1,2,4-triazol-3-yl]thio}pyridine; C₂₀H₁₅N₅O₃S; Sigma-Aldrich):

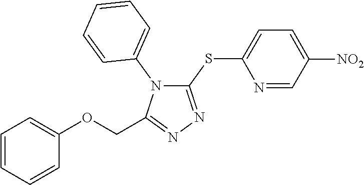 [SMILES: O=[N+]([O-])c1ccc(Sc2nnc(COc3ccccc3)n2-c2ccccc2)nc1]

The compounds provided herein can be synthesized using conventional techniques using readily available starting materials or the compounds may be purchased from commercial providers. MIND4-17, for example, is also known as 5-Nitro-2-{[5-(phenoxymethyl)-4-phenyl-4H-1,2,4-triazol-3-yl]thio}pyridine, having the molecular formula C₂₀H₁₅N₅O₃S, can be obtained from Sigma-Aldrich.

### Compositions

The methods described herein can be implemented through the use of compositions comprising a MIND4 compound, including pharmaceutical compositions, in which the MIND4 compound described herein serves as an active ingredient.

Pharmaceutical compositions typically include a pharmaceutically acceptable carrier. Such salts may be prepared by conventional means from the corresponding compound by reacting, for example, the appropriate acid or base with a compound described herein. Preferably the salts are in crystalline form, and preferably prepared by crystallization of the salt from a suitable solvent. A person skilled in the art will understand how to prepare and select suitable salt forms for example, as described in Handbook of Pharmaceutical Salts: Properties, Selection, and Use By P. H. Stahl and C. G. Wermuth (Wiley-VCH 2002).

A pharmaceutical composition is typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, solutions or suspensions used for parenteral, intradermal, or subcutaneous application can include the following components: a sterile diluent such as water for injection, saline solution, fixed oils, polyethylene glycols, glycerine, propylene glycol, or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates, or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

Pharmaceutical compositions suitable for injection can include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (for example, glycerol, propylene glycol, liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions can also be prepared in the form of suppositories (e.g., with conventional suppository bases such as cocoa butter and other glycerides) or retention enemas for rectal delivery.

Additionally, intranasal delivery is possible, as described in, inter alia, Hamajima et al., Clin. Immunol. Immunopathol., 88(2), 205-10 (1998). Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In one embodiment, the compounds are prepared with carriers that will protect the compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially. e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical composition may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. Concentrations and dosage values may also vary with the severity of the infection to be alleviated. It is to be further understood that for any particular patient, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Dosage forms or compositions containing a compound as described herein in the range of 0.005% to 100% with the balance made up from non-toxic carrier may be prepared. Methods for preparation of these compositions are known to those skilled in the art. The contemplated compositions may contain 0.001%-100% active ingredient, in one embodiment 0.1-95%, in another embodiment 75-85%.

In some embodiments, the dose of compound is about 20 to about 100 mg/kg body weight. In some embodiments, the dose of compound is about 40 to about 80 mg/kg body weight. In some embodiments, the dose of compound is about 60 mg/kg body weight.

The pharmaceutical compositions can be included in a container, pack, or dispenser together with instructions for administration.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### Methods

Described herein is a method of inhibiting viral replication in a cell infected with a coronavirus. In some embodiments, the method is a method of inhibiting viral replication in a cell infected with severe acute respiratory syndrome corona virus 2 (SARS-CoV-2). In some embodiments, the method comprises contacting the infected cell with a compound selected from the group consisting of the MIND4 compounds depicted in FIGS. 1A-1F, or a pharmaceutically acceptable salt of any one of these compounds. In some embodiments, the compound is

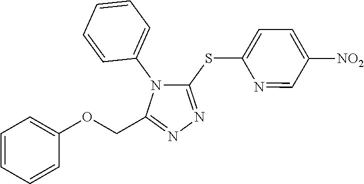 [SMILES: O=[N+]([O-])c1ccc(Sc2nnc(COc3ccccc3)n2-c2ccccc2)nc1]

or a pharmaceutically acceptable salt thereof.

In some embodiments, the cell is a neuron. In some embodiments, the neuron is in the central nervous system of a subject. In some embodiments, the cell is a lung cell. In some embodiments, the cell is ex vivo. In some embodiments, the cell is in vivo.

Also described herein is a method of treating a coronavirus infection in a subject. In some embodiments, the coronavirus infection is a SARS-CoV-2 infection. In some embodiments, the method comprises administering a therapeutically effective amount of the MIND4 compound recited above (or in FIG. 1A-1F) to the subject. Additionally described is a method of ameliorating inflammatory and/or immune reactions in a subject infected with a virus. In some embodiments, the method comprises administering a therapeutically effective amount of the MIND4 compound to the subject. In some embodiments, the administering is intravenous or intranasal. In some embodiments, the administering is by inhalation.

The subject is typically a mammal. In one embodiment, the mammal is human. In other embodiments, the mammal is a veterinary subject. Examples of veterinary subjects include, but are not limited to, equine, canine, bovine, porcine, ovine, and feline subjects.

### EXAMPLES

The following examples are presented to illustrate the present invention and to assist one of ordinary skill in making and using the same. The examples are not intended in any way to otherwise limit the scope of the invention.

### Example 1: MIND4-17 Inhibits Viral Growth in SARS-CoV-2-Infected Human Neurons

We have previously described a novel small molecule, MIND4 and its derivative MIND4-17 as an activator of Nrf2 and regulator of inflammatory responses in the context of Huntington's disease^{6,7}. (Small Molecule Activators of NRF2 Pathway U.S. Pat. No. 9,737,525 B2, incorporated by reference herein). These molecules are triazole-containing inducers of NRF2. MIND4 and MIND4-17 are highly selective for NRF2 as its mechanism of action is by covalently modifying a critical stress-sensor cysteine (C151) of the E3 ligase substrate adaptor protein Kelch-like ECH-associated protein 1 (KEAP1), the primary negative regulator of NRF2. In this Example, we evaluated the ability of MIND4-17 to modulate SARS-CoV2 infection in human induced pluripotent stem cell-derived neurons 8 and reduce inflammatory responses.

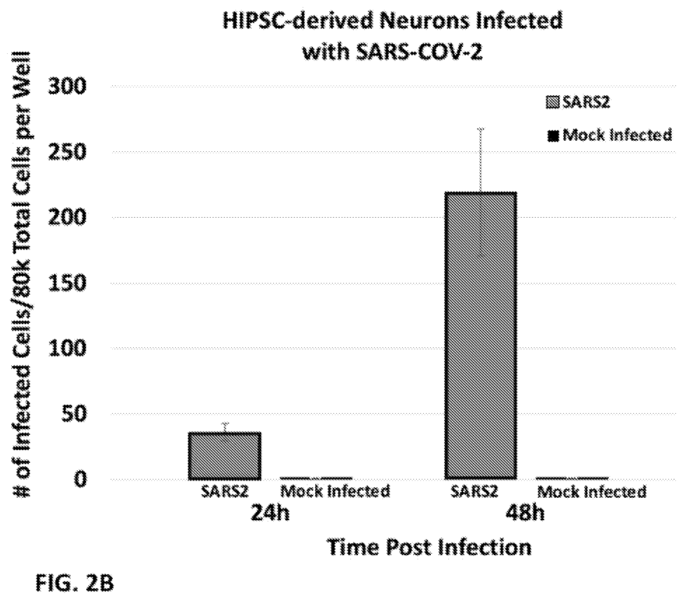

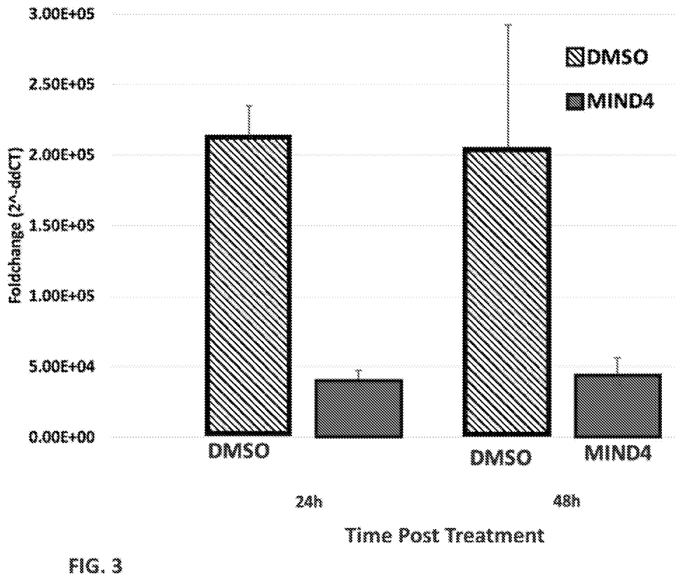

Numerous studies have indicated SARS-CoV-2 is capable of infecting neurons following exposure, and this may be linked to the diverse neurologic symptoms. To directly test this, we generated high-purity human iPSC-derived neurons and infected these cells with SARS-CoV-2 (Seattle strain) at a multiplicity of infection (MOI) of 0.1 and the ability of virus to infect cells determined by immunohistochemical staining for anti-nucleocapsid (N) staining of cells. As shown in FIG. 2A, we can detect SARS-CoV-2 N protein at both 24 and 48 hours post-infection (hpi) via immunocytochemical staining and show there is an increase in viral antigen by 48 hpi, as determined by counting positive cells/field (FIG. 2B), or progression of the condition or disease.

We next tested whether treatment of SARS-CoV-2 infected human neurons with MIND4-17 (2 uM final concentration) affected viral growth. Neurons were infected with SARS-CoV-2 (MOI=0.1) and treated with either MIND4-17 or vehicle (DMSO) beginning at 24 hpi. RNA was subsequently isolated from infected neurons treated with either DMSO or MIND4-17 at 48 and 72 hpi (24 and 48 hrs post-treatment) and SARS-CoV-2 levels determined by qPCR for Spike mRNA. As shown in FIG. 3, MIND4-17 treatment resulted in a dramatic reduction in Spike transcripts showing that MIND4-17 treatment effectively reduces viral replication in neurons.

### Example 2: MIND 4-17 Prevents Viral Entry Via Inhibition of the Tubulin Pathway

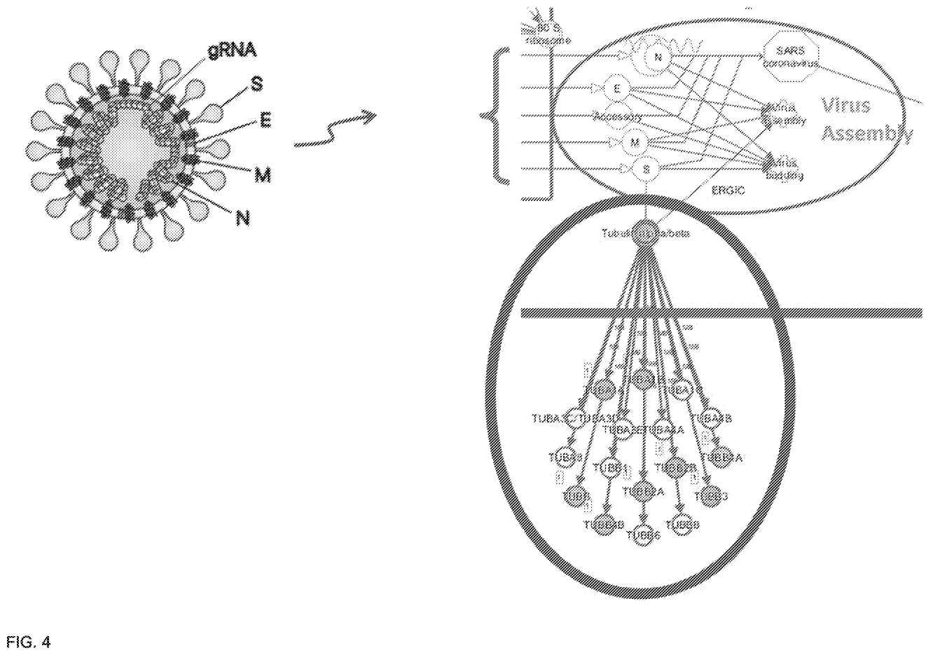

As a next step, sequencing was carried out on mRNA isolated from neurons infected with virus for 24 hours and either treated with vehicle (DMSO) or treated with MIND4-17 (see above). Hierarchical clustering and analysis of differentially expressed genes using Ingenuity Pathway Analysis (IPA) highlighted a novel mechanism for NRF2 activation in modulating viral responses. Per IPA's new Coronavirus Replication Pathway. Tubulins facilitate successful SARS-CoV2 viral assembly. These tubulins are highly overrepresented as downregulated in the mRNAseq in the presence of MIND 4-17 (FIG. 4), suggesting that MIND 4-17 at least in part prevents viral entry via inhibition of virus assembly via the tubulin pathway. This is consistent with ChIP-Seq data showing that tubulins can be a target of NRF2 through global profiling of NRF2 binding sites (Malhotra et al, Nucleic Acids Res, 2010.385718-34).

FIG. 4 illustrates how the tubulin network is required for SarsCoV2 viral assembly, with the tubulin network shown using IPA network-based analysis. One of the top networks discovered using IPA is this same tubulin network, showing that MIND4-17 causes downregulation of multiple genes in the network (shown in shaded circles) suggesting this as a mechanism of action for the anti-viral properties of MIND4.

See Rüdiger, A. T., et al., Tubulins interact with porcine and human S proteins of the genus Alphacoronavirus and support successful assembly and release of infectious viral particles. Virology (2016), 497:185-197, which relates to tubulins and coronaviruses.

### Example 3: In Vitro Testing of MIND4-17 Blocking of SARS-CoV-2 Replication in Human Cells

Cells e.g. human iPSC-derived neurons (described above) and human lung epithelial cell lines, specifically, Calu-3 cells (available from the ATCC), are incubated in 6-well plates at a concentration of 1×10⁶ cells/well. Cells are incubated at 37° C. in a cell incubation chamber for 24 hrs, at which point SARS-CoV-2 is added [multiplicity of infection (MOI)=0.1] to each well and virus allowed to adhere for 1 hr (rocking plates every 15 min) followed by washing and adding media back containing either DMSO (vehicle) or MIND4-17 (2 μM). Supernatants and cellular RNA were collected at 72 h post-infection to measure viral titers via plaque assay and quantitative PCR (qPCR—using primers specific for Spike RNA), respectively.

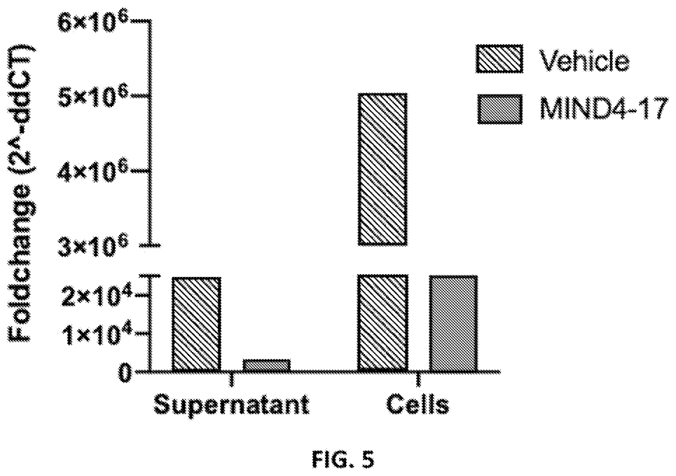

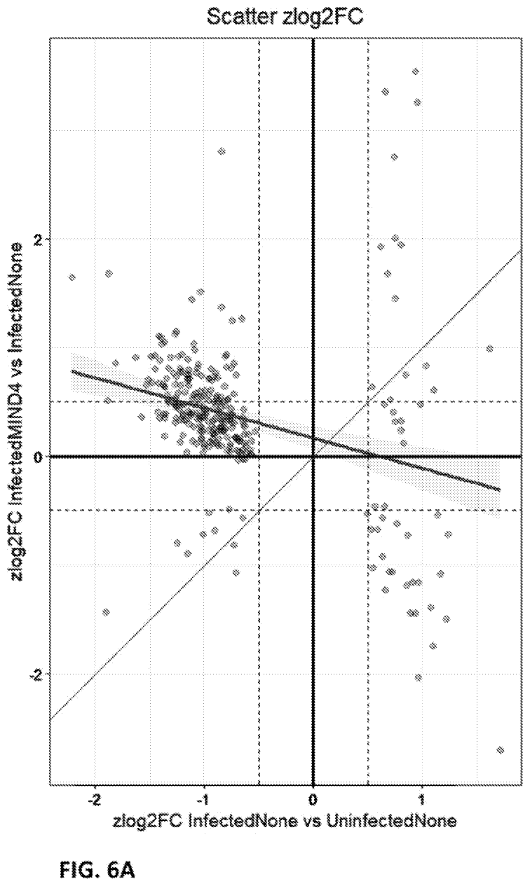

To evaluate the ability of MIND4 to impact SARS-CoV-2 replication in the human lung epithelial cell line, Calu3, confluent monolayers of cells were incubated at 37° C. in a cell incubation chamber for 24 hrs, at which point SARS-CoV-2 was added [multiplicity of infection (MOI)=0.1] to each well and virus allowed to adhere for 1 hr (rocking plates every 15 min) followed by washing and adding media back containing either DMSO or MIND4-17 (2 μm). Supernatants and cellular RNA were collected at 72 h post-infection to measure viral titers via plaque assay and quantitative PCR (qPCR—using primers specific for Spike RNA), respectively. RNA was also isolated to carry out total RNAseq. The results revealed that MIND4 treatment resulted in a dramatic reduction in viral RNA levels in Calu3-cells when compared to control-treated cells, confirming that MIND4-targeting impacts viral replication (FIG. 5).

Sequencing was next carried out on mRNA isolated from the treated and untreated Calu3 cells infected. The raw counts were subset to infected samples with no treatment and uninfected samples with no treatment to determine the gene expression patterns from viral infection. The samples were then analyzed with DESeq2 to identify DEGs. The raw counts were also subset to only infected samples treated with MIND4 and infected samples with no treatment and then analyzed with DESeq2 to evaluate gene expression changes exerted by MIND4. Genes passing an FDR of 10% were used for GO enrichment analysis using GOrilla and IPA with infection showing immune responses and MIND4 inhibition showing effects on cell cycle, DNA damage, and other responses.

Finally, to evaluate whether MIND4 treatment of infected cells could restore gene expression back to control, uninfected levels, z-scores were calculated for the log 2 fold changes of the genes in the “infected vs uninfected” and “infected+mind4 vs infected” comparisons seen on following z-plot. The lists were subset to only overlapping genes with FDR<10% for both comparisons and the log 2 fold change z-scores were plotted using ggplot2.

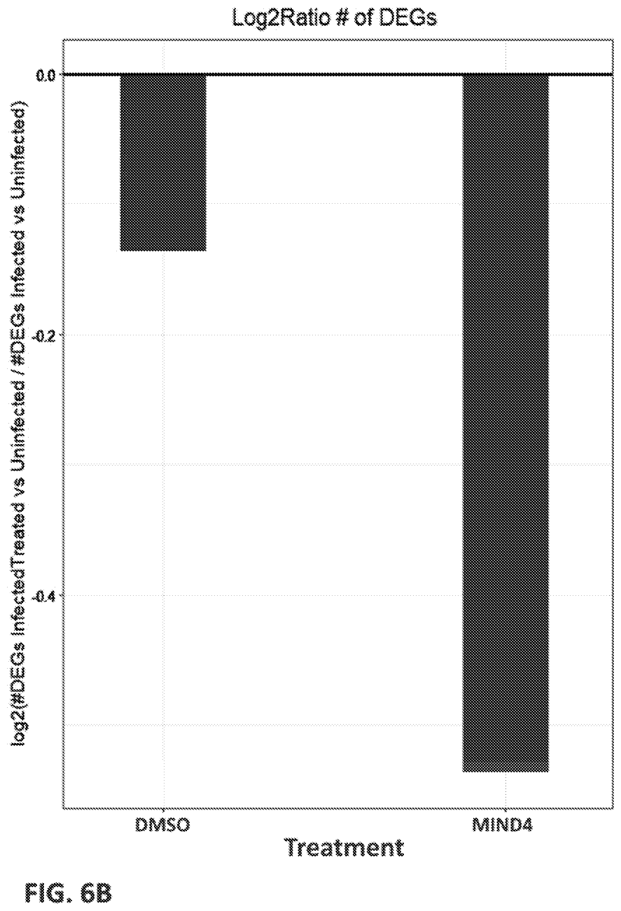

Remarkably, MIND4 infection showed a significant restoration back to uninfected state, as seen on the upper left quadrant on the scatter plot where the majority of genes showed reversal from the infected state (see FIGS. 6A-6B).

### REFERENCES

1 Kesic, M. J., Simmons, S. O., Bauer, R. & Jaspers, I. Nrf2 expression modifies influenza A entry and replication in nasal epithelial cells. Free Radic Biol Med 51, 444-453, doi:10.1016/j.freeradbiomed.2011.04.027 (2011).
2 Kosmider, B. et al. Nrf2 protects human alveolar epithelial cells against injury induced by influenza A virus. Respir Res 13, 43, doi:10.1186/1465-9921-13-43 (2012).
3 McCord, J. M., Hybertson, B. M., Cota-Gomez, A., Geraci, K. P. & Gao, B. Nrf2 Activator PB125((R)) as a Potential Therapeutic Agent against COVID-19. Antioxidants (Basel) 9, doi:10.3390/antiox9060518 (2020).
4 Hassan, S. M. et al. The Nrf2 Activator (DMF) and Covid-19: Is there a Possible Role? Med Arch 74, 134-138, doi:10.5455/medarh.2020.74.134-138 (2020).
5 Cuadrado, A. et al. Can Activation of NRF2 Be a Strategy against COVID-19?Trends Pharmacol Sci 41, 598-610, doi:10.1016fj.tips.2020.07.003 (2020).
6 Quinti, L. et al. SIRT2- and NRF2-Targeting Thiazole-Containing Compound with Therapeutic Activity in Huntington's Disease Models. Cell Chem Biol 23, 849-861, doi:10.10161j.chembiol.2016.05.015 (2016).
7 Quinti, L. et al. KEAP1-modifying small molecule reveals muted NRF2 signaling responses in neural stem cells from Huntington's disease patients. Proc Natl Acad Sci USA 114, E4676-E4685, doi:10.1073/pnas.1614943114 (2017).
8 Smith-Geater, C. et al. Aberrant Development Corrected in Adult-Onset Huntington's Disease iPSC-Derived Neuronal Cultures via WNNT Signaling Modulation. Stem Cell Reports 14, 406-419, doi:10.1016/j.stemcr.2020.01.015 (2020).

Throughout this application various publications are referenced. The disclosures of these publications in their entireties are hereby incorporated by reference into this application in order to describe more fully the state of the art to which this invention pertains.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A method of inhibiting viral replication in a cell infected with severe acute respiratory syndrome corona virus 2 (SARS-CoV-2), the method comprising contacting the infected cell with a compound selected from the group consisting of: 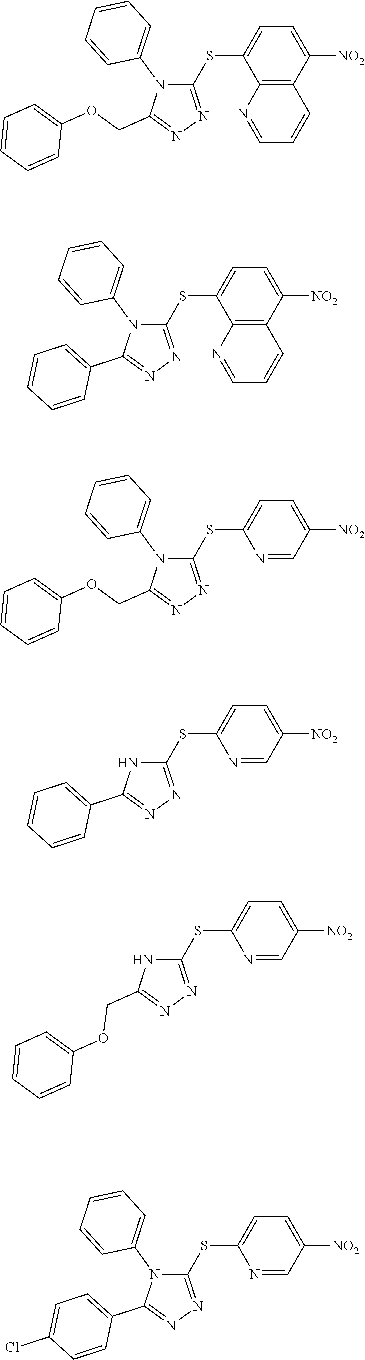 [SMILES: O=[N+]([O-])c1ccc(Sc2nnc(COc3ccccc3)n2-c2ccccc2)c2ncccc12 ; O=[N+]([O-])c1ccc(Sc2nnc(-c3ccccc3)n2-c2ccccc2)c2ncccc12 ; O=[N+]([O-])c1ccc(Sc2nnc(COc3ccccc3)n2-c2ccccc2)nc1 ; O=[N+]([O-])c1ccc(Sc2nnc(-c3ccccc3)[nH]2)nc1 ; O=[N+]([O-])c1ccc(Sc2nnc(COc3ccccc3)[nH]2)nc1 ; O=[N+]([O-])c1ccc(Sc2nnc(-c3ccc(Cl)cc3)n2-c2ccccc2)nc1]
or a pharmaceutically acceptable salt of any one thereof.

2. The method of claim 1, wherein the compound is: 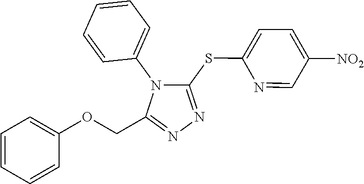 [SMILES: O=[N+]([O-])c1ccc(Sc2nnc(COc3ccccc3)n2-c2ccccc2)nc1]
or a pharmaceutically acceptable salt thereof.

3. The method of claim 1, wherein the cell is a neuron.

4. The method of claim 3, wherein the neuron is in the central nervous system of a subject.

5. The method of claim 1, wherein the cell is a lung cell.

6. The method of claim 1, wherein the cell is ex vivo.

7. The method of claim 1, wherein the cell is in vivo.

8. A method of treating a SARS-CoV-2 infection in a subject, the method comprising administering a therapeutically effective amount of the compound recited in claim 1 to the subject.

9. A method of ameliorating inflammatory and/or immune reactions in a subject infected with a virus, the method comprising administering a therapeutically effective amount of the compound recited in claim 1 to the subject.

10. The method of claim 8, wherein the administering is intravenous or intranasal.

11. The method of claim 8, wherein the administering is by inhalation.

12. The method of claim 7, wherein the compound is administered at a dose of about 40 to about 80 mg/kg body weight.

13. The method of claim 12, wherein the dose is about 60 mg/kg body weight.

14. The method of claim 9, wherein the administering is intravenous or intranasal.

15. The method of claim 9, wherein the administering is by inhalation.

16. The method of claim 8, wherein the compound is administered at a dose of about 40 to about 80 mg/kg body weight.

17. The method of claim 16, wherein the dose is about 60 mg/kg body weight.

18. The method of claim 9, wherein the compound is administered at a dose of about 40 to about 80 mg/kg body weight.

19. The method of claim 18, wherein the dose is about 60 mg/kg body weight.
